(19) United States
(12) Patent Application Publication
(10) Pub. No.: US 2025/0124305 A1
(43) Pub. Date: Apr. 17, 2025

(54) DESIGNING BACTERIAL COMMUNITIES USING MACHINE LEARNING

(71) Applicant: The University of Chicago, Chicago, IL (US)

(72) Inventors: Arjun Raman, Chicago, IL (US); Seppe Kuehn, Chicago, IL (US); Ana Rita Almeida de Oliveira, Chicago, IL (US)

(21) Appl. No.: 18/914,398

(22) Filed: Oct. 14, 2024

Related U.S. Application Data
(60) Provisional application No. 63/543,788, filed on Oct. 12, 2023.
(60) Provisional application No. 63/559,101, filed on Feb. 28, 2024.
(60) Provisional application No. 63/705,152, filed on Oct. 9, 2024.

(51) Int. Cl. G06N 3/126; G16B 20/40

(52) U.S. Cl. CPC G06N 3/126; G16B 20/40

(57) ABSTRACT

Methods, systems, and apparatus, including computer programs encoded on a computer storage medium, for training a machine learning model that is configured to process a model input that defines a bacterial community to generate a predicted score that predicts a performance of the bacterial community in performing a bacterial task. According to one aspect, a method comprises: generating data identifying a set of bacterial communities; obtaining, for each bacterial community, a target score for the bacterial community; generating a set of training examples, wherein each training example corresponds to a respective bacterial community and comprises: (i) a training input that identifies the bacterial strains included in the bacterial community, and (ii) the target score for the bacterial community; training the machine learning model on the set of training examples; and identifying one or more candidate bacterial communities for performing the bacterial task using the trained machine learning model.

### CROSS-REFERENCE TO RELATED APPLICATIONS

This application claims priority to U.S. Patent Application No. 63/543,788, filed on Oct. 12, 2023, U.S. Patent Application No. 63/559,101, filed on Feb. 28, 2024, and U.S. Patent Application No. 63/705,152, filed on Oct. 9, 2024. The disclosure of the foregoing applications is incorporated herein by reference in its entirety for all purposes.

### TECHNICAL FIELD

This specification relates to designing bacterial communities using machine learning.

### BACKGROUND

Bacteria are single-celled organisms that inhabit virtually every environment on Earth. They are an essential part of many ecosystems, and play important roles in processes such as nutrient cycling, decomposition, and the production of food, medicines, and other products. A bacterial cell has a nucleus containing genetic material, ribosomes for protein production, and mechanisms for energy production and waste disposal. Bacteria can exist as individual organisms or in communities, where they interact with each other and their environment. Bacteria can be adaptable and can survive and function in a wide range of environments, from extreme temperatures to highly acidic or salty conditions.

Machine learning models receive an input and generate an output, e.g., a predicted output, based on the received input. Some machine learning models are parametric models and generate the output based on the received input and on values of the parameters of the model.

### SUMMARY

This specification describes a system implemented as computer programs on one or more computers in one or more locations that can design bacterial communities for performing a bacterial task.

Throughout this specification, a “bacterial strain” refers to a type of bacteria having a particular genetic profile.

Throughout this specification, a “bacterial community” refers to a set of one or more bacterial strains. A bacterial community can include any appropriate number of bacterial strains, e.g., 1, 10, 100, or 1000 bacterial strains.

Throughout this specification a “physically synthesized instance” of a bacterial community refers to a real-world population of bacteria that instantiates the bacterial community, e.g., that includes one or more bacteria from each bacterial strain that is included in the bacterial community. The bacterial strains in a physically synthesized instance of a bacterial community can be represented within the physically synthesized instance in any appropriate proportions, e.g., in equal or unequal proportions.

Throughout this specification, a “bacterial task” can refer to any task that can be performed by a bacterial community. A bacterial community can be said to “perform” a bacterial task if a population of bacteria that includes bacteria from each bacterial strain in the bacterial community can contribute to accomplishing the task. A few examples of bacterial tasks are described next.

In some cases, a bacterial task can include achieving a therapeutic effect in a subject, e.g., a human or animal subject, e.g., by suppressing or enhancing a population of bacteria in the subject. For instance, a bacterial task can include suppressing a population of bacteria that can be harmful to human or animal health, e.g., Klebsiella pneumoniae, Salmonella, Staphylococcus aureus, Escherichia coli, Pseudomonas aeruginosa, or Clostridium difficile. As another example, a bacterial task can include enhancing a population of bacteria that can be beneficial for human health, e.g., Streptococcus thermophiles.

In some cases, a bacterial task can include an environmental remediation task, e.g., the degradation of contaminants in environmental media such as soil, groundwater, sediment, or surface water. For instance, a bacterial task can include the breakdown of Bisphenol A (BPA, a chemical compound that is used in the manufacture of various plastics), pesticides, petroleum products, or asbestos.

In some cases, a bacterial task can include facilitating (e.g., catalyzing) an industrial process, e.g., a process for manufacturing antibiotics, probiotics, drugs, vaccines, starter cultures, insecticides, enzymes, fuels, or solvents.

According to a first aspect there is provided a method performed by one or more computers, the method comprising: training a machine learning model that is configured to process a model input that defines a bacterial community to generate a predicted task score that predicts a performance of the bacterial community in performing a bacterial task, comprising: generating data identifying a set of bacterial communities, wherein each bacterial community comprises a plurality of bacterial strains; obtaining, for each bacterial community, a task score for the bacterial community that represents a performance of a physically synthesized instance of the bacterial community on the bacterial task; generating a set of training examples, wherein each training example corresponds to a respective bacterial community and comprises: (i) a training input that identifies the bacterial strains included in the bacterial community, and (ii) the task score for the bacterial community; training the machine learning model on the set of training examples; and identifying one or more bacterial communities for performing the bacterial task using the trained machine learning model.

In some implementations, generating data identifying the set of bacterial communities comprises generating data identifying the set of bacterial communities using operations that encourage genetic diversity of the plurality of bacterial strains included in each bacterial community.

In some implementations, the method further comprises obtaining data identifying a set of bacterial strains, wherein each bacterial strain is associated with a respective feature representation; wherein for one or more of the bacterial communities in the set of bacterial communities, generating the bacterial community comprises, at each of a plurality of iterations in a sequence of iterations: identifying a plurality of new bacterial strains, from the set of bacterial strains, that are not currently included in the bacterial community; determining, for each of the plurality of new bacterial strains, a distance between: (i) a feature representation of the new bacterial strain, and (ii) a respective feature representation of each of one or more bacterial strains currently included in the bacterial community; and selecting one or more of the new bacterial strains for inclusion in the bacterial community at the iteration based on the distances.

In some implementations, for each bacterial strain in the set of bacterial strains, the feature representation of the bacterial strain comprises a plurality of genetic features of the bacterial strain.

In some implementations, for each bacterial strain in the set of bacterial strains, the feature representation of the bacterial strain comprises a plurality of orthologous gene group features of the bacterial strain.

In some implementations, each bacterial community in the set of bacterial communities comprises bacterial strains selected from a set of bacterial strains; and the method further comprises: obtaining a matrix representing the set of bacterial strains; and performing dimensionality reduction on the matrix representing the set of bacterial strains.

In some implementations, performing dimensionality reduction on the matrix representing the set of bacterial strains reduces a number of bacterial strains in the set of bacterial strains.

In some implementations, the matrix representing the set of bacterial strains comprises a respective feature representation of each bacterial strain in the set of bacterial strains.

In some implementations, for each bacterial strain in the set of bacterial strains, the feature representation of the bacterial strain comprises a plurality of genetic features of the bacterial strain.

In some implementations, performing dimensionality reduction on the matrix representing the set of bacterial strains reduces a number of features in the respective feature representation of each bacterial strain.

In some implementations, identifying one or more bacterial communities for performing the bacterial task using the trained machine learning model comprises: generating a set of bacterial communities; generating a respective predicted task score for each bacterial community of the set of bacterial communities, comprising: processing a model input that defines the bacterial community using the machine learning model to generate the predicted task score for the bacterial community; and identifying one or more bacterial communities for performing the bacterial task using the predicted task scores.

In some implementations, identifying one or more bacterial communities for performing the bacterial task using the predicted task scores comprises: filtering the set of bacterial communities based on the predicted task scores, comprising removing a plurality of bacterial communities having lowest predicted task scores from the set of bacterial communities; generating a respective impact score for each bacterial strain in a set of bacterial strains using the set of bacterial communities; and identifying one or more bacterial communities for performing the bacterial task based at least in part on the impact scores for the bacterial strains.

In some implementations, generating a respective impact score for each bacterial strain in the set of bacterial strains using the set of bacterial communities comprises, for each bacterial strain: generating a matrix representing the set of bacterial communities, wherein the matrix comprises a respective feature representation of each bacterial community in the set of bacterial communities; processing the matrix representing the set of bacterial communities to generate a set of target vectors; and determining the impact score for the bacterial strain based on a projection of a vector representing the bacterial strain onto the set of target vectors.

In some implementations, processing the matrix representing the set of bacterial communities to generate the set of target vectors comprises: processing the matrix representing the set of bacterial communities to generate a set of latent vectors representing axes of data variance of the matrix; and identifying the set of target vectors as a proper subset of the set of latent vectors that have a highest statistical correlation with task scores for the bacterial task.

In some implementations, the method further comprises: generating a respective strain-strain covariance score for each pair of bacterial strains in the set of bacterial strains; and generating a set of candidate bacterial communities based on the strain-strain covariance scores; wherein identifying one or more bacterial communities for performing the bacterial task comprises: identifying one or more bacterial communities for performing the bacterial task based at least in part on: (i) the impact scores for the bacterial strains, and (ii) the set of candidate bacterial communities.

In some implementations, generating a respective strain-strain covariance score for each pair of bacterial strains in the set of bacterial strains comprises, for each pair of bacterial strains: determining the strain-strain covariance score for the pair of bacterial strains based on a similarity measure between: (i) a projection of a vector representing a first bacterial strain from the pair of bacterial strains onto a set of target vectors, and (ii) a projection of a vector representing a second bacterial strain from the pair of bacterial strains onto the set of target vectors.

In some implementations, generating a set of candidate bacterial communities based on the strain-strain covariance scores comprises: clustering the strain-strain covariance scores to identify a set of clusters of strain-strain covariances scores; and generating a respective candidate bacterial community corresponding to each cluster of strain-strain covariance scores, comprising, for each cluster of strain-strain covariances scores: generating a candidate bacterial community that includes each bacterial strain associated with a strain-strain covariance score in the cluster of strain-strain covariance scores.

In some implementations, identifying one or more bacterial communities for performing the bacterial task based at least in part on: (i) the impact scores for the bacterial strains, and (ii) the set of candidate bacterial communities, comprises: generating a respective selection score for each candidate bacterial community based at least in part on the impact scores for the bacterial strains; and identifying one or more of the candidate bacterial communities as bacterial communities for performing the bacterial task using the selection scores for the candidate bacterial communities.

In some implementations, identifying one or more bacterial communities for performing the bacterial task using the predicted task scores comprises: identifying one or more bacterial communities having highest predicted task scores from among the set of bacterial communities as bacterial communities for performing the bacterial task.

In some implementations, the set of bacterial strains comprise bacterial strains obtained from a fecal sample.

In some implementations, the set of bacterial strains comprise bacterial strains obtained from a soil sample.

In some implementations, the bacterial task comprises suppressing a target bacterial population.

In some implementations, for each bacterial community, the task score for the bacterial community is based at least in part on an abundance of the target bacterial population after the target bacterial population is co-cultured with the bacterial community.

In some implementations, the target bacterial population comprises Klebsiella pneumoniae.

In some implementations, the bacterial task comprises environment remediation.

In some implementations, the bacterial task comprises degrading an environmental contaminant.

In some implementations, for each bacterial community, the task score for the bacterial community is based at least in part on an abundance of the contaminant in the environment after the bacterial community is introduced into the environment.

In some implementations, the contaminant comprises Bisphenol A or byproducts of Bisphenol A.

In some implementations, the model input to the machine learning model comprises a numerical representation of the bacterial community that defines, for each bacterial strain in a collection of bacterial strains, whether the bacterial strain is included in the bacterial community.

In some implementations, training the machine learning model on the set of training examples comprises, for each training example: training the machine learning model to process the training input of the training example to generate a predicted task score that matches the task score specified by the training example.

In some implementations, the machine learning model comprises a random forest model.

In some implementations, the machine learning model comprises a neural network model.

In some implementations, the method further comprises, for each of one or more of the bacterial communities identified for performing the machine learning task: physically synthesizing an instance of the bacterial community.

In some implementations, the method further comprises, for each of one or more of the bacterial communities identified for performing the machine learning task: applying a physically synthesized instance of the bacterial community for performing the bacterial task.

According to another aspect, there is provided a method performed by one or more computers, the method comprising: obtaining data identifying a bacterial community; and generating a score that predicts a performance of the bacterial community in performing a bacterial task, comprising: generating a numerical representation of the bacterial community that defines, for each bacterial strain in a collection of bacterial strains, whether the bacterial strain is included in the bacterial community; and processing the representation of the bacterial community using a machine learning model to generate the score that predicts the performance of the bacterial community in performing the bacterial task.

In some implementations, the numerical representation of the bacterial community comprises a respective component for each bacterial strain in the collection of bacterial strains, wherein: any components corresponding to a bacterial strain that is included in the bacterial community have a first value in the numerical representation of the bacterial community; and any components corresponding to a bacterial strain that is not included in the bacterial community have a second value in the numerical representation of the bacterial community.

In some implementations, the first value is 1.

In some implementations, the second value is 0 or −1.

In some implementations, the machine learning model comprises a random forest model.

In some implementations, the machine learning model comprises a neural network model.

In some implementations, the machine learning model has been trained by operations comprising: obtaining a set of training examples, wherein each training example corresponds to a respective bacterial community and comprises: (i) a training input that identifies the bacterial strains included in the bacterial community, and (ii) the task score for the bacterial community; training the machine learning model on the set of training examples.

In some implementations, training the machine learning model on the set of training examples comprises, for each training example: training the machine learning model to process the training input of the training example to generate a predicted task score that matches the task score specified by the training example.

In some implementations, the bacterial task comprises suppressing a target bacterial population.

In some implementations, for each bacterial community, the task score for the bacterial community is based at least in part on an abundance of the target bacterial population after the target bacterial population is co-cultured with the bacterial community.

In some implementations, the target bacterial population comprises Klebsiella pneumoniae.

In some implementations, the bacterial task comprises environment remediation.

In some implementations, the bacterial task comprises degrading an environmental contaminant.

In some implementations, for each bacterial community, the task score for the bacterial community is based at least in part on an abundance of the contaminant in the environment after the bacterial community is introduced into the environment.

In some implementations, the contaminant comprises Bisphenol A or byproducts of Bisphenol A.

According to another aspect there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

Designing bacterial communities holds immense potential for addressing problems faced in environmental and human health settings. However, deriving principles of bacterial community design is daunting due to the complexity of microbiome-environment interactions. More specifically, the behavior and functions of bacterial communities emerge from the complex patterns of interactions between their constituent microbes and the environment. Elucidating mechanisms of action that underlie collective function becomes rapidly intractable as the size of a bacterial community increases and, even if achieved, is generally dependent on the environmental context in which community design was performed. As such, deeply characterizing individual microbes and then rationally combining bacteria is resource intensive and rarely produces the collective effect expected from the behavior of individual bacteria.

To address this issue, the system described in this specification can design bacterial communities for performing bacterial tasks without requiring mechanistic knowledge of individual bacterial strains. Rather, the system can automatically screen a space of possible bacterial communities using a machine learning model that can process data defining a bacterial community to generate a prediction for a task score that defines the performance of the bacterial community on a bacterial task. The system can train the machine learning model using a machine learning training technique on data derived from experiments on physically-synthesized instances of bacterial communities, thereby enabling the machine learning model to learn to implicitly identify and leverage complex patterns and correlations in bacterial data to accurately predict task scores.

The space of possible bacterial communities is exponentially large. For instance, 2^{N}−1 possible bacterial communities can be formed from a strain bank of N bacterial strains. Generating training data for training the machine learning model can require performing real-world experiments using physically-synthesized instances of bacterial communities. Physically-synthesizing and experimenting on bacterial communities can be expensive, difficult, and time-consuming. However, the performance of a machine learning model can heavily depend on the quality and richness of the training data used for training the machine learning model. Moreover, screening an entire space of possible bacterial communities may be computationally infeasible, even using an automated process based on a machine learning model. Therefore efficiently training the machine learning model and using the machine learning model for screening can require an effective strategy for prioritizing bacterial communities. The system described in this specification can generate bacterial communities, e.g., for training or screening, that include genetically diverse sets of bacterial strains, e.g., to reduce the likelihood of functional redundancy within selected bacterial communities. Generating genetically diverse bacterial communities can enable the system to train the machine learning model and screen the space of possible bacterial communities more efficiently, and can increase the likelihood of the system identifying bacterial communities that achieve high performance on a bacterial task.

Bacterial communities that include large numbers of bacterial strains can be expensive and difficult to physically synthesize. Moreover, the performance of bacterial communities with large numbers of bacterial strains on a bacterial task may be sensitive to environmental context, e.g., as a result of complex and unpredictable interactions between the large number of bacterial strains and the environment. Therefore, it can be desirable to construct “sparse” bacterial communities, e.g., that include only the “core” bacterial strains necessary to effectively perform a bacterial task. The system described in this specification can efficiently identify sparse bacterial communities that can effectively perform bacterial tasks. In particular, for each pair of bacterial strains in a set of bacterial strains, the system can generate a strain-strain covariance score that characterizes a relationship of the pair of bacterial strains in relation to performing the bacterial task. The system can cluster the strain-strain covariance scores to identify “candidate” bacterial communities for performing the bacterial task that are sparse, and in particular, that include bacterial strains that share similarities in relation to performing the bacterial task, e.g., that may operate synergistically to perform the bacterial task.

The system described in this specification was used to identify a set of 15 bacterial strains that when combined into a community: (i) sustainably suppressed K. pneumoniae across various diverse in vitro environments, (ii) matched the clearance ability of a fecal microbial transplant (FMT) in a pre-clinically relevant mouse model of infection, (iii) was a safe intervention in vivo, (iv) could not be obviously deconstructed into a functional subset of strains, and (v) did not resemble the composition of natural human gut microbiotas.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example design system.

FIG. 2A is a flow diagram of an example process for generating a set of diverse bacterial communities.

FIG. 2B illustrates an example of generating a bacterial community by a process that encourages genetic diversity of the bacterial strains included in the bacterial community.

FIG. 3A is a flow diagram of an example process for training a machine learning model, over a sequence of update iterations, to predict bacterial task scores.

FIG. 3B illustrates training a machine learning model, over a sequence of update iterations, to predict bacterial task scores.

FIG. 4 is a flow diagram of an example process for training a machine learning model at a current update iteration.

FIG. 5 is a flow diagram of an example process for designing one or more bacterial communities for performing a bacterial task using a machine learning model.

FIG. 6 is a flow diagram of an example process for determining a respective impact score for each bacterial strain in a set of possible bacterial strains.

FIG. 7 is a flow diagram of an example process determining a set of candidate bacterial communities using strain-strain covariance scores.

FIG. 8A-FIG. 8D show examples of experimental results on the bacterial task of suppressing drug-resistant Klebsiella pneumoniae bacteria.

Like reference numbers and designations in the various drawings indicate like elements.

### DETAILED DESCRIPTION

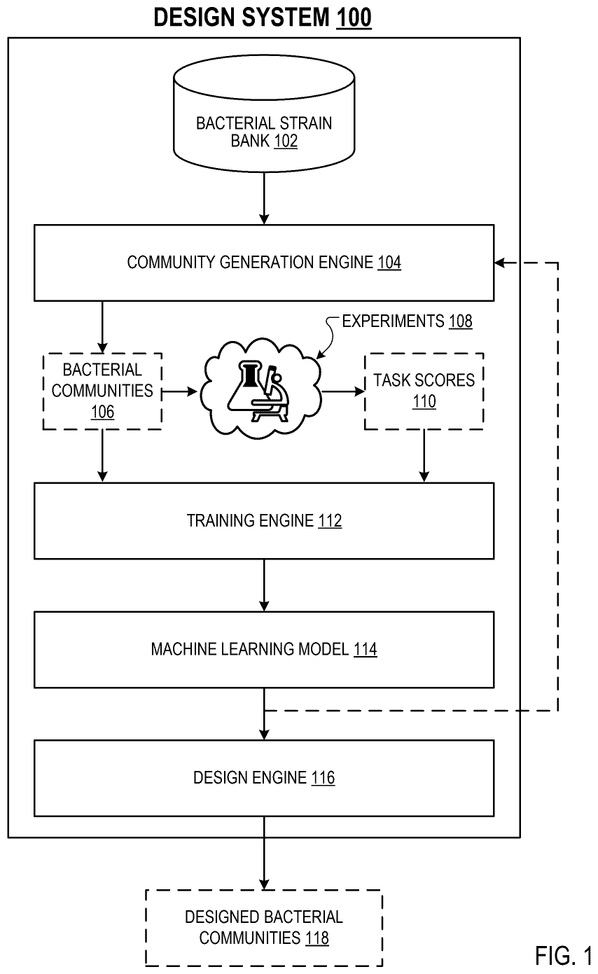

FIG. 1 shows an example design system 100. The design system 100 is an example of a system implemented as computer programs on one or more computers in one or more locations in which the systems, components, and techniques described below are implemented.

The design system 100 is configured to generate data identifying one or more bacterial communities 118 for performing a bacterial task. The design system 100 can include a bacterial strain bank 102, a community generation engine 104, a training engine 112, a machine learning model 114, and a design engine 116, which are each described in more detail next.

The strain bank 102 stores data identifying a set of bacterial strains. The strain bank 102 can include any appropriate number of bacterial strains, e.g., 10 bacterial strains, 100 bacterial strains, or 1000 bacterial strains. Data identifying a bacterial strain can include, e.g., data characterizing a genetic profile of the bacterial strain, e.g., data defining the genome sequence of the bacterial strain. The strain bank 102 can include bacterial strains obtained from any of a variety of possible sources. For instance, the strain bank 102 can include bacterial strains identified and isolated from soil samples, or water samples, or air samples, or fecal samples, or blood samples, or fluid samples, or tissue samples, etc.

Optionally, the design system 100 can filter the strain bank 102 to remove redundant bacterial strains, e.g., bacterial strains that have at least a threshold level of similarity with other bacterial strains included in the strain bank 102. The design system 100 can measure a level of similarity of two bacterial strains, e.g., by a comparison of the genetic sequences of the 16S ribosomal ribonucleic acid (RNA) of the bacterial strains. In a particular example, the design system 100 can remove a bacterial strain from the strain bank 102 in response to determining that the genetic sequence of the 16S ribosomal RNA of the bacterial strain is at least 98% identical to the genetic sequence of the 16S ribosomal RNA of another bacterial strain in the strain bank 102. Filtering the strain bank 102 to remove redundant bacterial strains can enable the design system 100 to more efficiently explore the space of possible bacterial communities.

The community generation engine 104 is configured to process data from the bacterial strain bank 102 to generate data identifying a set of bacterial communities 106. Each bacterial community 106 includes one or more bacterial strains from the strain bank 102; generally, some or all of the bacterial communities include multiple strains from the bacterial strain bank 102. The community generation engine 104 can generate any appropriate number of bacterial communities, e.g., 1000, 10,000, or 100,000 bacterial communities. The community generation engine 104 can generate bacterial communities 106 that include different numbers of bacterial strains. For instance, the community generation engine 104 can generate one bacterial community with 10 strains, and another bacterial community with 20 strains. The community generation engine 104 can generate the bacterial communities in any of a variety of possible ways. A few example techniques for generating bacterial communities are described next.

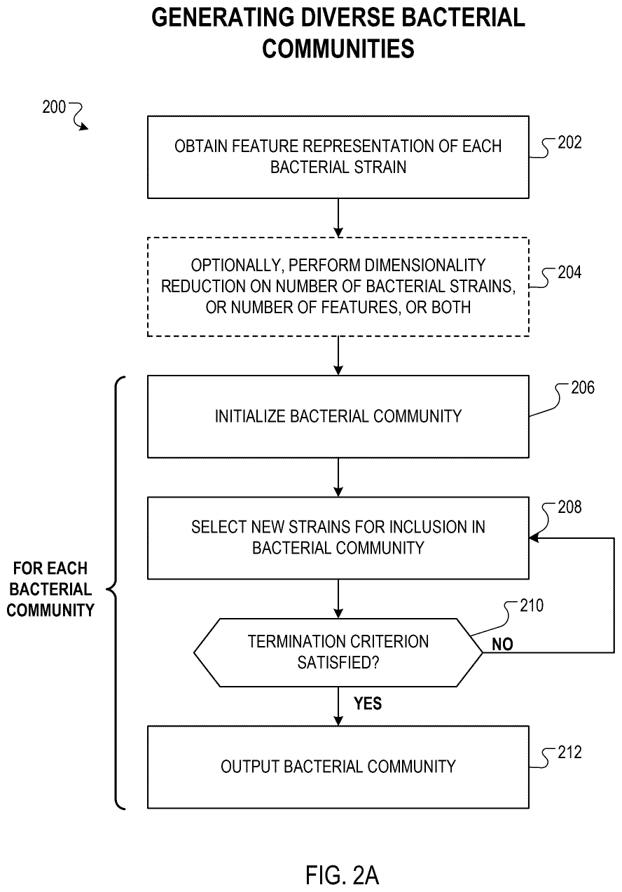

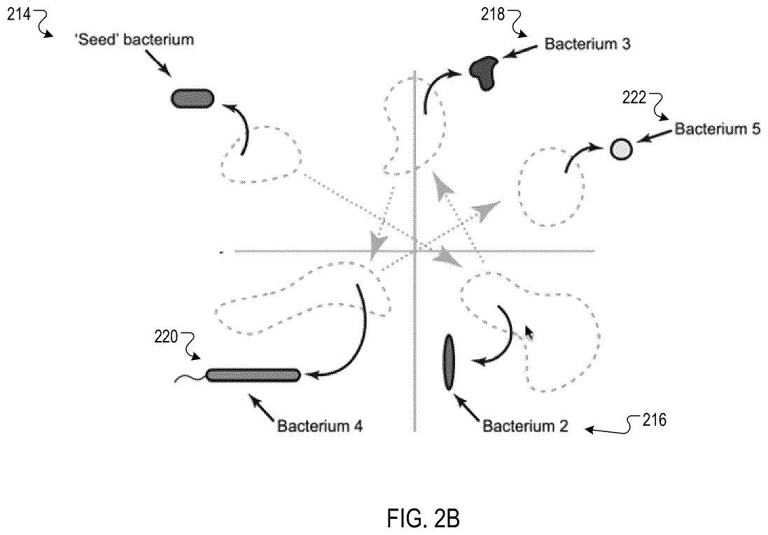

In some implementations, the community generation engine 104 generates bacterial communities 106 in a manner that encourages diversity, e.g., genetic diversity, of the bacterial strains included within each bacterial community. Increasing diversity within a bacterial community can reduce the likelihood of functional redundancy within the bacterial community. An example process for generating bacterial communities in a manner that encourages diversity within each bacterial community is described in more detail with reference to FIG. 2A.

In some implementations, to generate each bacterial community 106, the community generation engine 104 determines the number of bacterial strains to be included in the bacterial community, e.g., by sampling a number from a predefined probability distribution, e.g., a uniform distribution over a range of positive integer values. The community generation engine 104 can then populate the bacterial community by sampling the selected number of bacterial strains from the bacterial strain bank 102, e.g., in accordance with a predefined probability distribution over the bacterial strains included in the bacterial strain bank 102, e.g., a uniform distribution.

After generating the set of bacterial communities 106, the design system obtains a respective task score 110 for each bacterial community 106. The task score 110 for a bacterial community represents the performance of a physically synthesized instance of the bacterial community on the bacterial task (e.g., as determined through one or more experiments 108). More specifically, to generate a task score 110 for a bacterial community, an instance of the bacterial community is physically synthesized (e.g., using appropriate laboratory techniques), and the synthesized instance of the bacterial community is applied to perform the bacterial task. The performance of the synthesized instance of the bacterial community is measured, e.g., using an appropriate measurement technique, and the task score 110 for the bacterial community 106 is determined based on the measured performance of the synthesized instance of the bacterial community on the bacterial task. A few examples of determining a task score 110 for a bacterial community 106 for particular bacterial tasks are described next.

In some implementations, the bacterial task can be to suppress a target population of bacteria (e.g., bacteria that are harmful to human or animal health). To determine the task score 110 for a bacterial community 106, a physically synthesized instance of the bacterial community can be introduced into the environment of the target population of bacteria, e.g., in vivo (e.g., in an animal subject), or in vitro. The abundance of the target population of bacteria can be measured, e.g., using an appropriate biological assay, at one or more time points. The task score 110 for the bacterial community can then be determined based on, e.g., an abundance of the target population of bacteria measured at a particular time point. For instance, the task score 110 for the bacterial community can be determined as the percentage reduction in abundance of the target population of bacteria over a predefined time interval.

In a particular example, the bacterial task may be to suppress target bacteria harmful to human health, e.g., Klebsiella pneumoniae bacteria, or E. coli bacteria, or methicillin-resistant Staphylococcus aureus (MRSA) bacteria, or vancomycin-resistant Enterococci (VRE) bacteria, or Pseudomonas aeruginosa (MDR-PA) bacteria, or Acinetobacter baumannii (MDRAB) bacteria, or Clostridium difficile (C. diff) bacteria, or extended-spectrum beta-lactamase (ESBL) producing bacteria, or Mycobacterium tuberculosis (MDR-TB) bacteria, or Neisseria gonorrhoeae bacteria, or Streptococcus pneumoniae (MDRSP) bacteria. To determine the task score 110 for a bacterial community 106, a population of the target bacteria can be grown, e.g., to an particular optical density, e.g., an optical density of 0.6, and then co-cultured with a physically synthesized instance of the bacterial community 106 for 120 hours. The abundance of the target bacteria in the culture can be tracked by an appropriate assay, e.g., by a plate-based assay, and then used to determine the task score 110 for the bacterial community 106.

In some implementations, the bacterial task can be an environmental remediation task, e.g., to degrade contaminants in environmental media. To determine the task score 110 for a bacterial community 106, a physically synthesized instance of the bacterial community can be introduced into an environment that is includes one or more contaminants. The abundance of the contaminants can be measured, e.g., using an appropriate measurement technique, at one or more time points. The task score 110 for the bacterial community can then be determined, e.g., based on an abundance of the contaminants that remains after a predefined time interval, or based on a rate at which the bacterial community degrades the contaminants, or based on a combination (e.g., a linear combination) of these factors. In some cases, the task score 110 for the bacterial community can further account for, e.g., the initial concentration of the contaminants in the environment, as certain bacterial communities may perform better in environments with higher initial concentrations of contaminants.

In some implementations, the bacterial task can be an industrial task, e.g., to facilitate an industrial process, e.g., a process for manufacturing antibiotics, probiotics, drugs, vaccines, starter cultures, insecticides, enzymes, fuels, or solvents. To determine the task score 110 for a bacterial community 106, a physically synthesized instance of the bacterial community can be introduced into the relevant industrial process. The rate of output of the industrial process, or the quality (e.g., concentration) of output of the industrial process, can be measured at one or more time points using an appropriate measurement technique, and the task score 110 for the bacterial community 106 can be determined based on a combination of one or more of these factors.

The design system 100 can obtain the task scores 110 for the bacterial communities 106, e.g., from one or more users, by way of a user interface or an application programming interface (API) made available by the design system 100. More specifically, the design system 100 can provide data identifying the bacterial communities 106 generated by the community generation engine 104 to one or more users, e.g., by way of a user interface. Experiments 108 can be performed using physically synthesized instances of the bacterial communities 106, e.g., as described above, and the results of the experiments 108 can be provided to the design system 100 by way of a user interface or an API. The design system 100 can then determine a respective task score 110 for each bacterial community 106 based on the results of the experiments performed using the physically synthesized instance of the bacterial community, e.g., as described above. In some cases, one or more users can directly provide task scores 110 for the bacterial communities 106 to the design system 100, i.e., instead of providing the experimental results for processing by the design system 100 to derive task scores 110.

The design system 100 provides data identifying the bacterial communities 106 and their corresponding task scores 110 to the training engine 112. The training engine 112 is configured to train the machine learning model 114 on training data that represents the bacterial communities 106 and the corresponding task scores 110.

The machine learning model 114 is configured to receive a model input that includes a numerical representation of an input bacterial community. In particular, the model input defines, for each bacterial strain in a collection of bacterial strains, whether the bacterial strain is included in the input bacterial community. The machine learning model 114 is configured to process the model input, in accordance with values of a set of machine learning model parameters, to generate a predicted task score that predicts the performance of the input bacterial community in performing the bacterial task.

The model input to the machine learning model can numerically represent a bacterial community in any of a variety of possible ways. A few examples of numerical representations of bacterial communities are described next.

In some implementations, a bacterial community can be represented by an ordered collection of numerical values, e.g., a vector of numerical values, that includes a respective numerical value for each bacterial strain in a set of bacterial strains. (The set of bacterial strains can be, e.g., the set of bacterial strains included in the bacterial strain bank 102). The numerical value for a bacterial strain can define whether the bacterial strain is included in the bacterial community. For instance, a bacterial community can be represented vector of numerical values, where each bacterial strain that is included the bacterial community is represented by a first value (e.g., 1), and each bacterial strain that is not included in the bacterial community is represented by a second, different value (e.g., 0 or −1).

In some implementations, a bacterial community can be represented by a collection of embeddings. More specifically, each bacterial strain in a set of bacterial strains can be represented by a respective embedding. The embedding representing a bacterial strain can be, e.g., manually defined (e.g., in accordance with a heuristic), or learned jointly with the machine learning model 114 during training of the machine learning model 114. A bacterial community can be represented by a collection of embeddings that includes a respective embedding representing each bacterial strain that is included in the bacterial community. (Throughout this specification, an “embedding” refers to an ordered collection of numerical values, e.g., a vector, matrix, or other tensor of numerical values).

The machine learning model 114 can be any appropriate type of machine learning model, e.g., a random forest model, a neural network model, a support vector machine model, etc. Further, the machine learning model 114 can have any appropriate machine learning model architecture that enables the machine learning model 114 to perform its described functions. For instance, in an implementation where the machine learning model 114 is implemented as a random forest model, the random forest model can include any appropriate number of decision trees, having any appropriate depth, and using any appropriate splitting function at each node of each decision tree. As another example, in an implementation where the machine learning model 114 is implemented as a neural network model, the neural network model can include any appropriate types of neural network layers (e.g., fully connected layers, attention layers, etc.) in any appropriate numbers (e.g., 5 layers, 10 layers, or 15 layers) and connected in any appropriate configuration (e.g., as a linear sequence of layers or as a directed graph of layers).

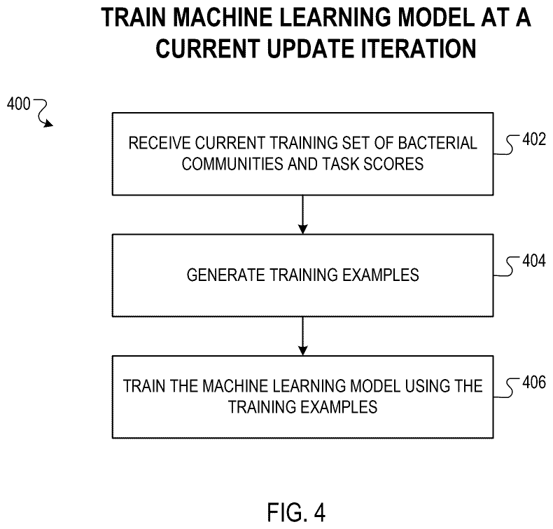

The training engine 112 trains the machine learning model 114 on training data representing the bacterial communities 106 and the corresponding task scores 110 using a machine learning training technique. An example process for training the machine learning model 114 is described in more detail with reference to FIG. 4.

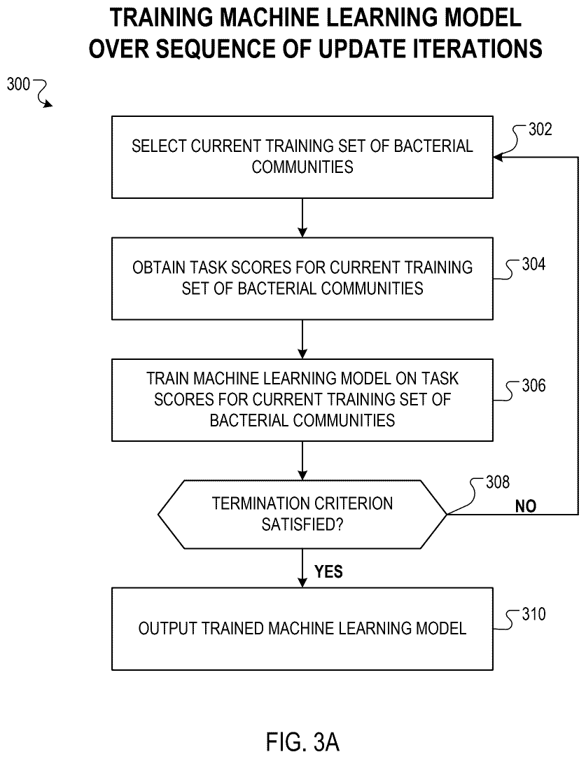

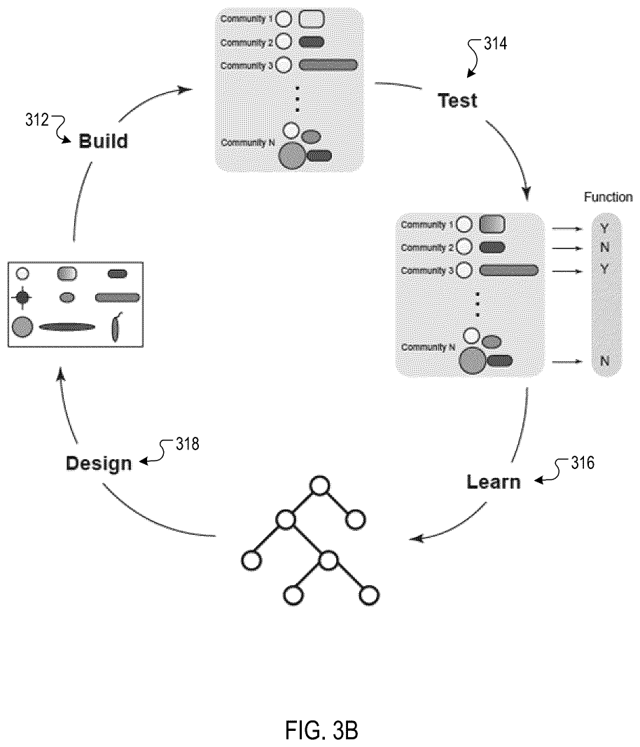

Optionally, the design system 100 can train the machine learning model over a sequence of multiple update iterations. At each update iteration, the design system 100 can generate new training data, and then update the machine learning model 114 using the new training data. The design system 100 can continue performing update iterations until the design system 100 determines that a termination criterion has been satisfied. The termination criterion can be, e.g., that the design system 100 has performed a threshold number of update iterations on the machine learning model 114, or that the machine learning model 114 has achieved at least a threshold level of accuracy. An example process for training the machine learning model over a sequence of multiple update iterations is described in more detail with reference to FIG. 3A.

The design system 100 can provide the trained machine learning model 114 (e.g., after the last update iteration has been performed) to the design engine 116, and the design engine 116 can use the trained machine learning model 114 to identify one or more bacterial communities 118 for performing the bacterial task. A few example techniques by which the design engine 116 can use the machine learning model 114 to identify bacterial communities for performing the bacterial task are described next.

In some implementations, the design system 100 can generate a collection of bacterial communities, e.g., at least 10,000, or 100,000, or 1,000,000 bacterial communities (e.g., using the community generation engine 104), and generate a respective predicted task score for each bacterial community using the machine learning model 114. The design system 100 can then select one or more bacterial communities having the highest predicted task scores as being the bacterial communities 118 for performing the bacterial task. For instance, the design system 100 can select a predefined number of the bacterial communities having the highest predicted task scores, or the design system 100 can select each bacterial community having a predicted task score that satisfies (e.g., exceeds) a threshold.

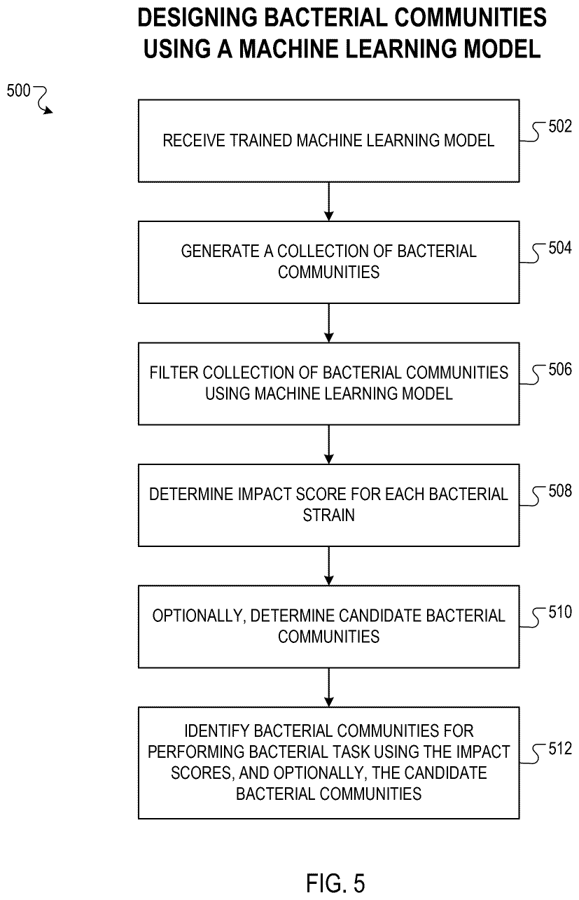

In some implementations, the design system 100 can use the machine learning model 114 to generate a respective impact score for each bacterial strain that characterizes a predicted contribution of the bacterial strain to performing the machine learning task. Optionally, the design system 100 can further generate a respective strain-strain covariance score for each pair of bacterial strains that characterizes a relationship between the pair of bacterial strains in relation to performing the bacterial task. The design system 100 can use the impact scores and the strain-strain covariance scores to identify sparse bacterial communities for performing the bacterial task, as will be described in more detail with reference to FIG. 5-FIG. 7.

The bacterial communities 118 generated by the design system 100 can be physically synthesized, and their effectiveness at performing the bacterial task can be experimentally validated. Bacterial communities 118 generated by the design system 100 that are demonstrated as being effective for performing the bacterial task can then be produced and deployed for the purpose of performing the bacterial task. For instance, if the bacterial task is to suppress a target population of bacteria that are harmful to human or animal health, then the bacterial communities can be incorporated into therapeutics (e.g., drugs), e.g., for treating bacterial infection by the target population of bacteria. As another example, if the bacterial task is an environmental remediation task, then the bacterial communities can be seeded in contaminated environments to degrade contaminants and contribute to restoring the environments. As another example, if the bacterial task is to facilitate an industrial process, then the bacterial communities can be incorporated into the industrial process, e.g., to increase efficiency or yields of the industrial process.

FIG. 2A is a flow diagram of an example process 200 for generating a set of diverse bacterial communities. For convenience, the process 200 will be described as being performed by a system of one or more computers located in one or more locations. For example, a design system, e.g., the design system 100 of FIG. 1, appropriately programmed in accordance with this specification, can perform the process 200.

The system obtains a respective feature representation of each bacterial strain in a collection of bacterial strains (202). For instance, the system can obtain a respective feature representation of each bacterial strain in the bacterial strain bank described with reference to FIG. 1. A feature representation of a bacterial strain refers to an ordered collection of features, e.g., a vector of features, characterizing the bacterial strain, e.g., characterizing the genetic characteristics of the bacterial strain.

The system can generate a respective feature representation of each bacterial strain, e.g., by processing data defining the genome sequence of the bacterial strain. For instance, the system can generate a feature representation of a bacterial strain that includes a respective feature corresponding to each orthologous gene group (OGG) in a collection of OGGs. The value of a feature corresponding to an OGG can represent a count of the OGG in the bacterial proteome of the bacterial strain. Alternatively or in combination with generating feature representations based on OGGs, the system can generate feature representations using any of a variety of other possible features, e.g., features derived from gene ontology, Search Tool for the Retrieval of Interacting Genes/Proteins (STRING) pathways, methylation context sensitive enzyme ddRAD (MCSeEd) pathways, etc.

Optionally, the system can perform dimensionality reduction on the number of bacterial strains in the collection of bacterial strains, or on the feature representations of the bacterial strains, or both (204).

Performing dimensionality reduction on the number of bacterial strains in the collection of bacterial strains can involve replacing the original collection of bacterial strains by a collection of “clustered” bacterial strains. Each clustered bacterial strain can represent a group of one or more bacterial strains from the original collection of bacterial strains. Some or all of the clustered bacterial strains represent multiple bacterial strains from the original collection of bacterial strains, such that the collection of clustered bacterial strains includes fewer bacterial strains than the original collection of bacterial strains. For instance, the number of clustered bacterial strains can be less than the number of original bacterial strains, e.g., by a factor of 2×, or 5×, or 10×, or 100×. For convenience, the clustered bacterial strains (i.e., resulting from performing dimensionality reduction of the number of bacterial strains) are also referred to throughout this specification as bacterial strains.

Performing dimensionality reduction on the feature representations of the bacterial strains can involve generating a new feature representation for each bacterial strain that includes fewer features than the original feature representation of the bacterial strain. The new feature representation for a bacterial strain can include rich features than encode some or all of the information content of the original (larger) feature representation of the bacterial strain. The number of features in the new feature representations of the bacterial strains can be less than the number of features in the original feature representations of the bacterial strains, e.g., by a factor of 2×, or 5×, or 10×, or 100×.

The system can perform dimensionality reduction on the number of bacterial strains, or on the feature representations of the bacterial strains, or both using any appropriate dimensionality reduction technique. In some cases, the dimensionality reduction technique can operate on a matrix that represents the collection of bacterial strains, e.g., where each row of the matrix corresponds to a respective bacterial strain and where each column of the matrix corresponds to a respective bacterial strain feature. The system can perform the dimensionality reduction using any appropriate dimensionality reduction technique, e.g., a Uniform Manifold Approximation and Projection for Dimension Reduction (UMAP) dimensionality reduction technique, a principal component analysis (PCA) dimensionality reduction technique, a t-distributed stochastic neighbor embedding (t-SNE) dimensionality reduction technique, or a singular value decomposition (SVD) dimensionality reduction technique.

The system can use the dimensionality-reduced collection of bacterial strains for generating one or more diverse bacterial communities, as will be described next with reference to steps 206-212. Performing dimensionality reduction can dramatically reduce the complexity of the space of possible bacterial communities. For instance, the number of possible bacterial communities that can be constructed from a set of N bacterial strains is 2^{N}−1. The original number of bacterial strains may number in the thousands, resulting in a computationally intractably large number of possible bacterial communities. Reducing the number of bacterial strains by one or more orders of magnitude, e.g., to tens or hundreds of bacterial strains, can significantly reduce the complexity of the design space of possible bacterial communities. Further, reducing the dimensionality of the feature representations of the bacterial strains can enable more efficient numerical comparison of feature representations of different bacterial strains, which can reduce the computational complexity of constructing diverse bacterial communities.

The system can perform the steps 206-210 to generate a bacterial community. The system can generate any appropriate number of bacterial communities, e.g., 100, 1,000, or 100,000 bacterial communities. For convenience, the following will describe performing the steps 206-210 to generate one bacterial community.

The system can initialize the bacterial community, e.g., by selecting one or more bacterial strains for initial inclusion in the bacterial community (206). For instance, the system can sample one or more bacterial strains for initial inclusion in the bacterial distribution from a probability distribution (e.g., a uniform probability distribution) over the collection of bacterial strains.

At each iteration in a sequence of iterations, the system can select one or more bacterial strains for inclusion in the bacterial community at the iteration (208). In particular, the system can identify a set of “candidate” bacterial strains that are not currently included in the bacterial community as of the iteration. The set of candidate bacterial strains can include, e.g., all of the bacterial strains not included in the bacterial community as of the iteration, or some subset of the set of bacterial strains not included in the bacterial community as of the iteration, e.g., selected by random sampling. In particular, for each of one or more “candidate” bacterial strains that are not currently included in the bacterial community, the system can determine a respective distance between: (i) the candidate bacterial strain, and (ii) each of one or more bacterial strains that are currently included in the bacterial community. The system can determine whether to include each candidate bacterial strain in the bacterial community based on the distances between the candidate bacterial strain and the bacterial strains currently in the bacterial community. The system can determine a distance between a first bacterial strain and a second bacterial strain, e.g., by evaluating a distance between a feature representation of the first bacterial strain and a feature representation of the second bacterial strain using any appropriate distance measure, e.g., an L₁ or L₂ distance measure.

The system can select bacterial strains for inclusion in the bacterial community at each iteration in order to encourage diversity (e.g., genetic diversity) between the bacterial strains included in the bacterial community. For instance, in one example implementation, the system can add bacterial strains to the bacterial community one at a time. More specifically, the system initializes the bacterial community with a single bacterial strain (at step 206) and adds a single bacterial strain at each iteration of step 208. The system can determine, for each candidate bacterial strain, a respective distance between: (i) the candidate bacterial strain, and (ii) the bacterial strain that was most recently added to the bacterial community. The system can select the candidate bacterial strain having the greatest distance from the bacterial strain that was most recently added to the bacterial community for inclusion in the bacterial community.

At each iteration in the sequence of iterations, the system determines whether a termination criterion is satisfied (210). The termination criterion can be, e.g., that the bacterial community includes a threshold number of bacterial strains. In response to determining that the termination criterion is not satisfied, the system returns to step 208. In response to determining that the termination criterion is satisfied, the system outputs the bacterial community.

In another implementation, to the system can initially generate M (e.g., M=10,000) bacterial communities of size N (e.g., N=20). The ensemble of all M communities of size N is represented as

C_{size N}={c₁, . . . ,c_{M}}

Each community, cᵢ, is defined by a set of N bacterial strains:

cᵢ={s₁, . . . ,s_{N}}

where sⱼ is strain j in cᵢ. The system computes all pairwise distances for all strains in Cᵢ. For instance, the pairwise distance between strain 1 and 2 may be:

pd_{1,2}=dist(s₁,s₂)

where ‘dist’ is the function that computes the distance between s₁ and s₂. The distribution of all pairwise distances for cᵢ may be defined as:

$$P {D}_{i} = \{ p {d}_{1 , 2} , {pd}_{1 , 3} , … , {pd}_{N - 1 , N} \}$$

The system orders PDᵢ for a given cᵢ from largest to smallest values, then computes the mean pairwise distance across the lower X % (e.g., X=30%) of values comprising PDᵢ. We term this value the ‘mean adjusted dispersal’.

The system computes the mean adjusted dispersal for all communities in C_{size N}.

The system then identifies the community within the M communities comprising C_{size N} with the maximum mean adjusted dispersal. This community is the designed community comprising N strains. The system can iteratively repeat this process to generate any number of bacterial communities with any number of strains.

FIG. 2B illustrates an example of generating a bacterial community by a process that encourages genetic diversity of the bacterial strains included in the bacterial community. The system can initially select a “seed” bacterial strain 214, then select a second bacterial strain 216 that is maximally distant from the seed bacterial strain, then select a third bacterial strain 218 that is maximally distant from the second bacterial strain, then select a fourth bacterial strain 220 that is maximally distant from the third bacterial strain, then select a fifth bacterial strain 222 that is maximally distant from the fourth bacterial strain, etc.

FIG. 3A is a flow diagram of an example process 300 for training a machine learning model, over a sequence of update iterations, to predict bacterial task scores. For convenience, the process 300 will be described as being performed by a system of one or more computers located in one or more locations. For example, a design system, e.g., the design system 100 of FIG. 1, appropriately programmed in accordance with this specification, can perform the process 300.

The system can perform the iteratively perform the steps 302-308 to train the machine learning model. Each iteration of the steps 302-308 is referred to as an update iteration. For convenience, the following will describe the steps 302-308 with reference to a “current” update iteration.

The system selects a current training set of bacterial communities for the current update iteration (302). The current training set of bacterial communities can include any appropriate number of bacterial communities, e.g., 10, 50, 100, or 1000 bacterial communities. The system can select the current training set of bacterial communities in any of a variety of possible ways. A few example techniques for selecting the current training set of bacterial communities are described next.

In some implementations, at each update iteration, the system can select the current training set of bacterial communities by generating a set of genetically diverse bacterial communities, e.g., in accordance with steps 206-212 of the process described with reference to FIG. 2.

In some implementations, at each update iteration after the first update iteration, the system can select the current training set of bacterial communities in a manner that is conditioned on the current performance of the machine learning model, i.e., as of the current update iteration. For instance, the system can generate a large set of “candidate” bacterial communities (e.g., 10,000, 50,000, or 100,000 candidate bacterial communities), e.g., in accordance with steps 206-212 of the process described with reference to FIG. 2. The system can then use the machine learning model to select a proper subset of the set of candidate bacterial communities as the current training set of bacterial communities.

For instance, to select the current training set of bacterial communities from the set of candidate bacterial communities, the system can generate a respective predicted task score for each candidate bacterial community using the machine learning model. More specifically, for each candidate bacterial community, the system can process a representation of the candidate bacterial community using the machine learning model, in accordance with current values of a set of model parameters of the machine learning model, to generate a respective predicted task score for the candidate bacterial community. Further, the system can generate a confidence score for each candidate bacterial community that represents a confidence of the machine learning model in the predicted task score generated for the candidate bacterial community. The system can select the current training set of bacterial communities from the set of candidate bacterial communities using the confidence scores, or the predicted task scores, or both.

In some cases, the system can select a number of candidate bacterial communities associated with the lowest confidence scores (from among the set of candidate bacterial communities) as the current training set of bacterial communities. For instance, the system can select a predefined number of candidate bacterial communities with the lowest confidence scores as the current training set of bacterial communities. As another example, the system can select each candidate bacterial community with a confidence score that satisfies (e.g., falls below) a threshold for inclusion in the current training set of bacterial communities.

In some cases, the system select the current training set of bacterial communities using both the confidence scores and the predicted task scores. For instance, the system can identify: (i) a “high-performing” set of candidate bacterial communities having predicted confidence scores that exceed a threshold; and (ii) a “low-performing” set of candidate bacterial communities having predicted confidence scores that are below a threshold. The system can then select: (i) a number of candidate bacterial communities having the lowest confidence scores from among the high-performing set of candidate bacterial communities, and (ii) a number of candidate bacterial communities having the lowest confidence scores from among the low-performing set of candidate bacterial communities, for inclusion in the current training set of bacterial communities.

Training the machine learning model on bacterial communities associated with low confidence scores can contribute to increasing the prediction accuracy of the machine learning model, e.g., by requiring the machine learning model to focus on difficult or ambiguous parts of the space of possible bacterial communities.

The system can determine confidence scores for that represent a confidence of the machine learning model in predicted task scores generated for bacterial communities in any of a variety of ways. One example technique for generating confidence scores for random forest models is described with reference to: Stefan Wager, Trevor Hastie, Bradley Efron, “Confidence intervals for random forests: the jackknife and the infinitesimal jackknife,” Journal of Machine Learning Research 15 (2014) 1625-1651.

The system obtains a respective task score for each bacterial community in the current training set of bacterial communities (304). The task score for a bacterial community can indicate a performance of a physically synthesized instance of the bacterial community on the bacterial task. Example techniques for generating task scores for bacterial communities are described in more detail with reference to FIG. 1.

The system trains the machine learning model using at least the task scores for the current training set of bacterial communities (306). An example process for training the machine learning model using the task scores for the current training set of bacterial communities is described in more detail with reference to FIG. 4.

The system determines whether a termination criterion is satisfied (308). The system can determine that a termination criterion is satisfied, e.g., if the system has performed a threshold number of update iterations, or if the machine learning model has achieved a threshold prediction accuracy. In response to determining that a termination criterion is not satisfied, the system can return to step 302 and perform another update iteration. In response to determining that a termination criterion has been satisfied, the system can output the trained machine learning model, e.g., for use in designing one or more bacterial communities to perform the bacterial task, as described with reference to FIG. 5.

FIG. 3B illustrates training a machine learning model, over a sequence of update iterations, to predict bacterial task scores. At each update iteration, the system can “build” 312 one or more bacterial communities, the bacterial communities can be “tested” 314 to determine task scores defining their effectiveness for performing the bacterial task, the machine learning model can “learn” 316 based on the task scores, and the machine learning model can be used to “design” 318 bacterial communities for use at the next update iteration.

FIG. 4 is a flow diagram of an example process 400 for training a machine learning model at a current update iteration. For convenience, the process 400 will be described as being performed by a system of one or more computers located in one or more locations. For example, a design system, e.g., the design system 100 of FIG. 1, appropriately programmed in accordance with this specification, can perform the process 400.

The system receives: (i) a current training set of bacterial communities, and (ii) a respective task score for each bacterial community in the current training set of bacterial communities (402). An example process for determining a current training set of bacterial communities and corresponding task scores is described with reference to FIG. 3. Optionally, if the current update iteration is after the first update iteration, the system can receive training sets of bacterial communities and corresponding task scores from one or more preceding update iterations.

The system generates a set of training examples (404). Each training example includes: (i) a training input to the machine learning model that identifies a respective bacterial community, and (ii) a target score corresponding to the training input. The system can generate the target scores for the training examples in any of a variety of ways. For instance, the system can define the target score for each training example as representing the task score for the bacterial community of the training example. As another example, the system can define the target score for each training example as represents a difference between: (i) the task score for the bacterial community, and (ii) a predicted task score for the bacterial community. The system can generate the predicted task score for the bacterial community using the machine learning model, i.e., in accordance with the current values of the set of model parameters of the machine learning model.

The system trains the machine learning model using the set of training examples by a machine learning training technique (406). A few example techniques for training the machine learning model using the set of training examples are described next.

In some implementations, the target score for each training example defines the task score for the bacterial community of the training example. The system can train the machine learning model on each training example, in particular, by training the machine learning model to process the training input of the training example to generate a predicted task score that matches the task score of the training example. If the machine learning model is of a type that enables iterative training, then the system can train the machine learning model by iteratively refining the current values of the set of machine learning model parameters, e.g., using stochastic gradient descent. Other types of machine learning models, e.g., random forest models, may not allow training through iterative refinement. In these cases, the system can generate and train a new machine learning model on the set of training examples, e.g., by training a new forest of decision trees on the set of training examples.

In some implementations, the target score for each training example can represent a difference between: (i) the task score for the bacterial community, and (ii) a predicted task score for the bacterial community. In these implementations, the machine learning model can be an ensemble model, i.e., that includes an ensemble of “constituent” machine learning models (which can each be, e.g., a decision tree, or a random forest, or a neural network, etc.). The output of the machine learning model can be a combination, e.g., a sum or weighted sum, of the outputs of the constituent machine learning models in the ensemble. At each update iteration, the system can train a new constituent machine learning model on the current set of training examples, and add the new constituent machine learning model to the ensemble of constituent machine learning models. The system can train a new constituent machine learning model on a training example by training the new constituent machine learning model to process the training input of the training example to generate a predicted score that matches the target score for the training example. Thus the system can train the new constituent machine learning model to learn to correct errors and biases in the existing ensemble of constituent machine learning models.

The system can train the machine learning model using any appropriate machine learning training technique appropriate for the architecture of the machine learning model. More specifically, the system can train the machine learning model to optimize an objective function. The objective function can measure, for each training example, an error between: (i) a predicted score (e.g., task score) generated by the machine learning model by processing the training input of the training example, and (ii) the target score of the training example. The objective function can measure the error between a predicted score and a target score, e.g., as a L₁ error, or an L₂ error, or using any other appropriate error metric.

The system can train the machine learning model to process a numerical representation of a bacterial community that defines, for each bacterial strain in a collection of bacterial strains, whether the bacterial strain is included in the bacterial community. An alternative approach may be to represent a bacterial community, e.g., based on a metabolite profile of the bacterial community. However, certain empirical results suggest that alternative representations of bacterial communities, e.g., based on metabolite profiles, may result in lower prediction accuracy than representations based on strain presence-absence. To understand why the metabolite profile of a community was a poor predictor of task performance (e.g., for the task of clearing K. pneumoniae), one may interrogate the structure of metabolite profiles across the bacterial communities used to train the machine learning model. The neighborhood of metabolite space where there were bacterial communities that achieved strong task performance also contained poorly performing bacterial communities. That is, the metabolic landscape of bacterial communities is ‘rugged’-interspersed with peaks and valleys of suppressive capacity-rather than smooth. This result demonstrates there is a degeneracy of different, unrelated metabolite profiles associated with bacterial task performance resulting in a predictive model that was overfit to the training set and therefore unable to generate new functional communities. Consistent with this result, bacterial communities that achieved high performance on the bacterial task shared similar metabolite profiles with bacterial communities that exhibited intermediate to low performance on the bacterial task.

In contrast, the landscape of bacterial communities defined by strain presence-absence is smooth. Thus, describing bacterial communities by their strain presence-absence defined a space that was co-linear with bacterial task performance and thereby enables learning an accurate statistical model of design. Collectively, these results indicate that, in some cases, design based on a metabolic profile comprising a targeted panel of features (amino acids, aromatics, branch-chained fatty acids, indoles, phenolic aromatics, and short-chained fatty acids) may not be a reliable strategy for engineering bacterial communities in a predictable manner.

FIG. 5 is a flow diagram of an example process 500 for designing one or more bacterial communities for performing a bacterial task using a machine learning model. For convenience, the process 500 will be described as being performed by a system of one or more computers located in one or more locations. For example, a design system, e.g., the design system 100 of FIG. 1, appropriately programmed in accordance with this specification, can perform the process 500.

The system receives a machine learning model that has been trained to process a model input that defines a bacterial community to generate a prediction for a task score for the bacterial community (502). The task score for the bacterial community represents the performance of a physically synthesized instance of the bacterial community on the bacterial task. An example process for training a machine learning model to generate predicted task scores is described with reference to FIG. 3A.

The system generates a collection of bacterial communities (504). Each bacterial community includes a set of bacterial strains, where each bacterial strain is selected from a set of possible bacterial strains. The set of possible bacterial strains can be, e.g., the set of strains included in a bacterial strain bank, e.g., as described with reference to FIG. 1. In some cases, the set of possible bacterial strains include clustered bacterial strains (each of which represent a respect “cluster,” i.e., group, of bacterial strains) that have been generated by performing dimensionality reduction on a set of possible bacterial strains, as described with reference to FIG. 2. The system can generate the collection of bacterial communities in any appropriate way. For instance, the system can generate a collection of genetically diverse bacterial communities using the operations described with reference to FIG. 2. The collection of bacterial communities can include any appropriate number of bacterial communities, e.g., 100,000, 1 million, or 10 million bacterial communities.

The system filters the collection of bacterial communities using the machine learning model (506). In particular, for each bacterial community, the system processes a model input that defines the bacterial community using the machine learning model, in accordance with trained values of a set of model parameters of the machine learning model, to generate a predicted task score for the bacterial community. The system then removes one or more bacterial communities from the collection of bacterial communities based on the predicted task scores. In particular, the system can remove one or more bacterial communities having the lowest task scores from among the collection of bacterial communities (where a lower task score is understood to represent a worse performance on the bacterial task). That is, the system can filter the collection of bacterial communities to remove a number of bacterial communities having the lowest performance on the bacterial task, i.e., as defined by the predicted task scores generated by the machine learning model. For instance, the system can remove each bacterial community having a task score that satisfies (e.g., falls below) a threshold.

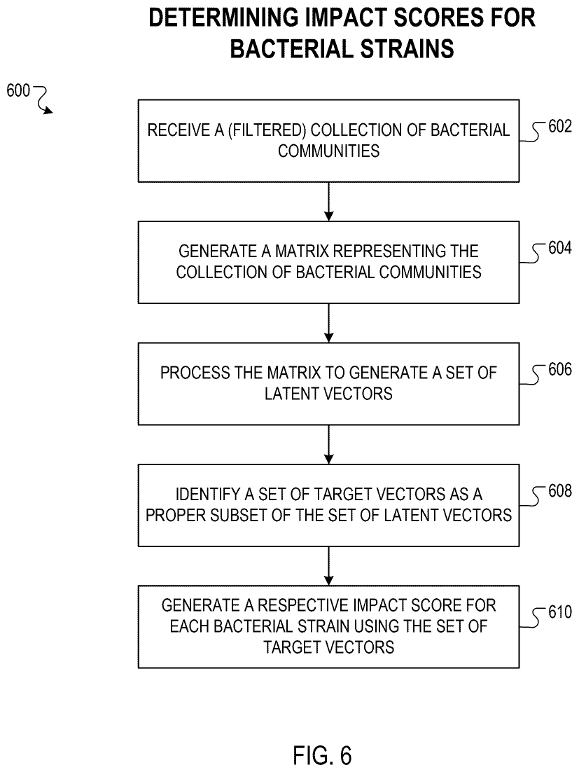

The system determines a respective impact score for each bacterial strain in the set of possible bacterial strains (508). An impact score for a bacterial strain characterizes a predicted contribution of the bacterial strain to performing the bacterial task. The system can determine the impact scores for the bacterial strains in any of a variety of ways. An example process for determining a respective impact score for each bacterial strain is described with reference to FIG. 6.

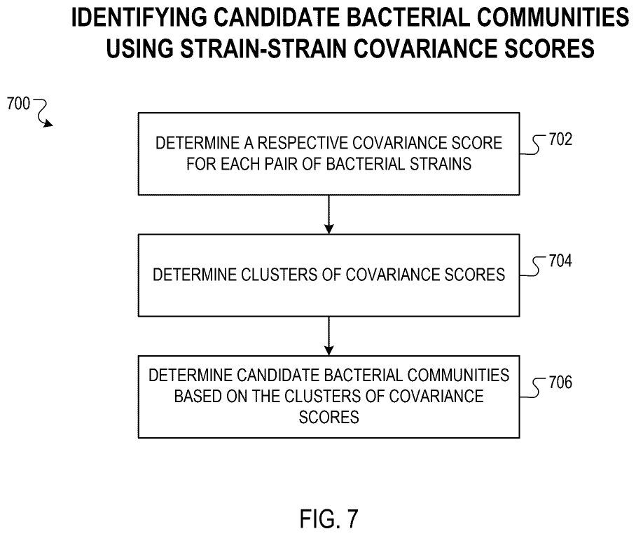

Optionally, the system can determine a set of candidate bacterial communities (510). To generate the set of candidate bacterial communities, the system can generate a respective strain-strain covariance score for each pair of bacterial strains. A covariance score for a pair of bacterial strains characterizes a relationship between the pair of bacterial strains in relation to performing the bacterial task. The system can determine the set of candidate bacterial communities such that covariance scores between pairs of bacterial strains included in the same candidate bacterial community tend to be more similar than covariance scores between pairs of bacterial strains included in different candidate bacterial communities. An example process for determining a set of candidate bacterial communities based on strain-strain covariance scores is described with reference to FIG. 7.

The system identifies one or more bacterial communities for performing the bacterial task using the impact scores, and optionally, the set of candidate bacterial communities (512).

In some implementations, the system can generate a selection score for each candidate bacterial community based on: (i) the impact scores for the bacterial strains included in the bacterial community, and optionally, (ii) covariance scores for pairs of bacterial strains included in the bacterial community. The system can then identify one or more of the candidate bacterial communities having the highest selection scores as bacterial communities for performing the bacterial task.

To generate a selection score for a candidate bacterial community, the system can generate an aggregate impact score for the candidate bacterial community based on the impact scores of the bacterial strains included in the candidate bacterial community. For instance, the system can generate the aggregate impact score as a measure of central tendency (e.g., a mean, median, or mode) of the impact scores of the bacterial strains included in the candidate bacterial community, or as the maximum or minimum of the impact scores of the bacterial strains included in the candidate bacterial community. Further, the system can generate a clustering score for the candidate bacterial community based on the covariance scores of pairs of bacterial strains included in the candidate bacterial community. For instance, the system can generate the clustering score for the candidate bacterial community based on (e.g., as an inverse of) a measure of dispersion (e.g., a variance) of the covariance scores between pairs of bacterial strains included in the bacterial community. The system can generate the selection score for the candidate community based on one or both of the aggregate impact score and the clustering score. For instance, the system can generate the selection score for the candidate community as a weighted linear combination of the aggregate impact score and the clustering score.

In some implementations, the system can identify one or more bacterial communities for performing the bacterial task using the impact scores alone, i.e., without reference to candidate bacterial communities generated using strain-strain covariance scores. For instance, the system can identify a number (e.g., a predefined number) of bacterial strains having the highest impact scores as being a bacterial community for performing the bacterial task.

FIG. 6 is a flow diagram of an example process 600 for determining a respective impact score for each bacterial strain in a set of possible bacterial strains. The impact score for a bacterial strain characterizes a predicted contribution of the bacterial strain to performing the bacterial task. For convenience, the process 600 will be described as being performed by a system of one or more computers located in one or more locations. For example, a design system, e.g., the design system 100 of FIG. 1, appropriately programmed in accordance with this specification, can perform the process 600.

The system receives a (filtered) collection of bacterial communities (602). An example process for generating a set of bacterial communities and filtering the set of bacterial communities to maintain only those bacterial communities that best perform the bacterial task is described with reference to steps 504-506 of FIG. 5.

The system generates a matrix representing the collection of bacterial communities (604). The system can generate the matrix by concatenating (row-wise or column-wise) a respective vector representing each bacterial community in the collection of bacterial communities. A vector can represent a bacterial community by representing, for each bacterial strain in a set of possible bacterial strains, whether the bacterial strain is included in the bacterial community. For instance, a vector representing a bacterial community can include a respective entry corresponding to each bacterial strain in the set of possible bacterial strains. Each entry of the vector that corresponds to a bacterial strain that is included in the bacterial community can have a first value (e.g., 1), and each entry of the vector that corresponds to a bacterial strain that is not included in the bacterial community can have a second value (e.g. 0 or −1).

The system processes the matrix to generate a set of latent vectors (606). The system can generate the set of latent vectors in any of a variety of possible ways. For instance, in some implementations, each latent vector can be a respective eigenvector of the matrix A, where A is the matrix representing the collection of bacterial communities. As another example, each latent vector can be a respective eigenvector of the matrix AAT, where A is the matrix representing the collection of bacterial communities and A^{T} is the transpose of A. As another example, each latent vector can represent a respective eigenvector of the matrix A^{T} A, where A and A^{T} are defined in the same way. The system can compute the eigenvectors, e.g., by way of a singular value decomposition, or in any other appropriate way. The latent vectors can, in some cases, be interpreted as representing the axes of data variance.

The system identifies a set of target vectors as a proper subset of the set of latent vectors (608). For instance, the system can identify the set of target vectors as the proper subset of the set of latent vectors that are most statistically correlated with the task scores.

In more detail, for each of one or more proper subsets of the set of latent vectors, the system determines a statistical correlation between: (i) the magnitude of vectors representing the bacterial communities when projected onto the subspace defined by the proper subset of the set of latent vectors, and (ii) the task scores for the bacterial communities. The system can measure the statistical correlation, e.g., as a Pearson correlation coefficient, or as a Spearman's rank correlation, or in any other appropriate way. The system can then identify the set of target vectors as being the proper subset of the set of latent vectors that is associated with the highest statistical correlation with the task scores for the bacterial communities. In some cases, the set of target vectors may include only a single latent vector.

In some implementations, the system can modify the task scores prior to determining the set of target vectors, e.g., in order to isolate “regress out” the effect of one or more auxiliary variables on the task scores. An auxiliary variable can be, e.g., the number of strains included in a bacterial community. To this end, the system can train a linear model to predict the task score as a function of the one or more auxiliary variables:

$$y = {\sum }_{i = 1}^{n} {β}_{i} {x}_{i} + {β}_{0}$$

where y is the task score for a bacterial community, (xᵢ)ᵢ₌₁ⁿ are the auxiliary variables (e.g., including the number of strains included in the bacterial community), and (βᵢ)ᵢ₌₀ⁿ are learned parameters of the linear model. For each bacterial community, the system can redefine the task score as a residual (difference) between: (i) the original task score for the bacterial community, and (ii) a predicted task score generated for the bacterial community by processing the one or more auxiliary variables for the bacterial community using the linear model.

The system generates a respective impact score for each bacterial strain using the set of target vectors (610). For instance, for each bacterial strain, the system can generate the impact score for the bacterial strain based on a magnitude of a projection of the vector representing the bacterial strain onto the subspace spanned by the set of target vectors.

FIG. 7 is a flow diagram of an example process 700 determining a set of candidate bacterial communities using strain-strain covariance scores. For convenience, the process 700 will be described as being performed by a system of one or more computers located in one or more locations. For example, a design system, e.g., the design system 100 of FIG. 1, appropriately programmed in accordance with this specification, can perform the process 700.

The system determines a respective covariance score for each pair of bacterial strains in the set of possible bacterial strains (702). A covariance score for a pair of bacterial strains characterizes a relationship between the pair of strains in relation to the bacterial task. That is, the system determines the covariance score for a pair of bacterial strains in a manner that is conditioned on (i.e., that depends on) the bacterial task. For instance, a covariance score for a pair of bacterial strains can characterize a similarity between the pair of bacterial strains in relation to their contribution to performing the bacterial task.

The system can determine covariance scores for pairs of bacterial strains in any of a variety of possible ways. A few example techniques for determining covariance scores for pairs of bacterial strains are described next.

In some implementations, the system determines the covariance scores for pairs of bacterial strains using a set of target vectors. The system generates the set of target vectors by generating a matrix representing the (filtered) collection of bacterial communities, processing the matrix to generate a set of latent vectors, and then identifying the set of target vectors as a proper subset of the set of latent vectors. For instance, the system can identify the set of target vectors as being a proper subset of the set of latent vectors that are most significantly correlated with the task scores for the bacterial task. An example process for generating a set of target vectors is described with reference to steps 602-608 of FIG. 6.

The system can generate a covariance score for a pair of bacterial strains (including a first bacterial strain and a second bacterial strain) based on a similarity measure between: (i) a projection of a vector representing the first bacterial strain onto the subspace spanned by the set of target vectors, and (ii) a projection of a vector representing the second bacterial strain onto the subspace spanned by the set of target vectors. The similarity measure can be any appropriate similarity measure, e.g., a similarity measure based on a Manhattan distance, or an L₁ distance, or an L₂ distance, etc. A bacterial strain can be represented as a vector in any of a variety of possible ways. For instance, a bacterial strain can be represented as a vector that includes a respective entry corresponding to each bacterial strain in the set of bacterial strains; the entry corresponding to the bacterial strain being represented can have a first value (e.g., 1), and the other entries can each have a second value (e.g., 0 or −1).

In more detail, let u∈ [symbol] ⁴⁶ be the vector of column projections of each bacterial strain onto the subspace spanned by the set of target vectors. Let s be the scalar value denoting the maximum value of u, and

$${A}^{46 \times 46} = ( {a}_{ij} ) \\ {a}_{ij} = {d}_{ij}^{2} = {‖ {u}_{i} - {u}_{j} ‖}^{2}$$

Where ∥·∥ denotes the Euclidian norm on [symbol] ⁴⁶

$${A}_{i , j} = [ \begin{matrix}0 & ⋯ & {d}_{46 , 1}^{1} \\ ⋮ & ⋱ & ⋮ \\ {d}_{1 , 46}^{2} & ⋯ & 0\end{matrix} ] \\ {S}_{i , j} = s - {A}_{i , j} = [ \begin{matrix}s & ⋯ & s - {d}_{46 , 1}^{2} \\ ⋮ & ⋱ & ⋮ \\ s - {d}_{1 , 46}^{2} & ⋯ & s\end{matrix} ]$$

The resulting symmetric similarity matrix, S_{i,j}, with rows and columns indicating each strain and each element representing the similarity between strain i and strain j describes how strains are related to one another based on their projections on the subspace defined by the set of target vectors.

In some implementations, the system determines the covariance scores for pairs of bacterial strains by training a polynomial regression model that can process data identifying a bacterial community to generate a predicted task score for the bacterial community. The polynomial regression model can have the form:

$$y = {β}_{0} + {\sum }_{i = 1}^{N} {β}_{i} {x}_{i} + {\sum }_{i , j = 1}^{N} {γ}_{i , j} {x}_{i} {x}_{j}$$

where y is the predicted task score for a bacterial community, xᵢ (for i=1 to N, where N is the number of bacterial strains in the set of bacterial strains) is a variable indicating whether strain i is included in the bacterial community, and β₀, (βᵢ), and (γ_{i,j}) are coefficients that are learned through training the polynomial regression model on a set of training examples, e.g., using a least-squares fitting method. Each training example can include: (i) a training input to the polynomial regression model that defines a bacterial community, and (ii) a task score for the bacterial community. Optionally, the polynomial regression model can have additional higher order terms. After training the polynomial regression model, the system can identify the covariance score for each pair of bacterial strains (i, j) as the value of the coefficient γ_{i,j} that scales a product of respective features indicating whether the bacterial strains in the bacterial community.

The system identifies a set of clusters of strain-strain covariance scores (704). The system can cluster the covariance scores using an appropriate clustering algorithm, e.g., a hierarchical agglomerative clustering algorithm or a k-means clustering algorithm. “Clustering” the covariance scores can refer to partitioning the set of covariance scores into a set of clusters (groups), where covariance scores included in the same cluster tend to be more similar than covariance scores included in different clusters.

The system determines a set of candidate bacterial communities based on the clusters of strain-strain covariance scores (706). In particular, for each cluster of covariance scores, the system can generate a candidate bacterial community that includes each bacterial strain associated with at least one covariance score included in the cluster of covariance scores. By construction, pairs of bacterial strains included in the same candidate bacterial community tend to have strain-strain covariance scores that are more similar than pairs of bacterial strains included in different candidate bacterial communities. Strains that are more similar are often found in communities that perform well on the bacterial task, and those that are more distant are more rarely found in communities that perform well on the bacterial task.

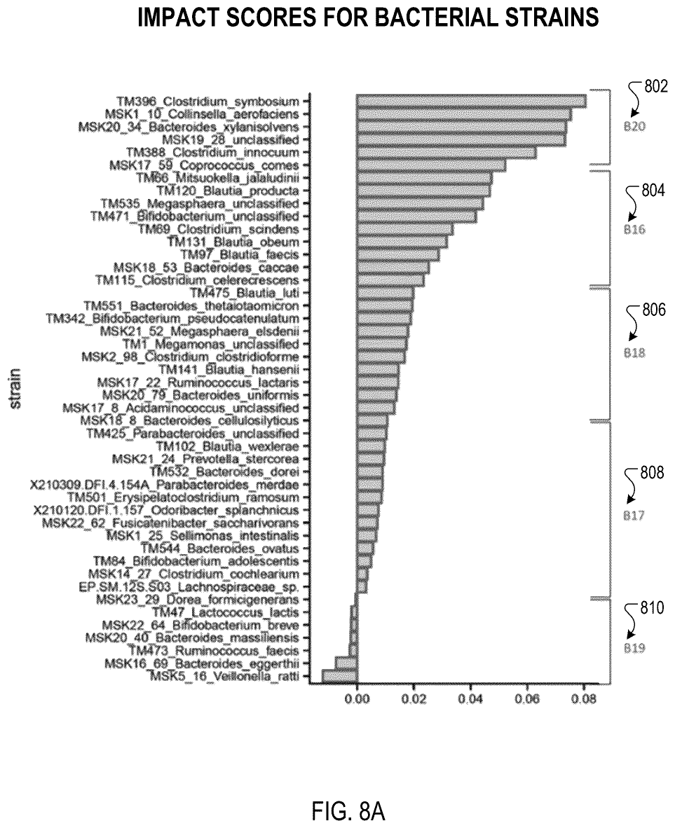

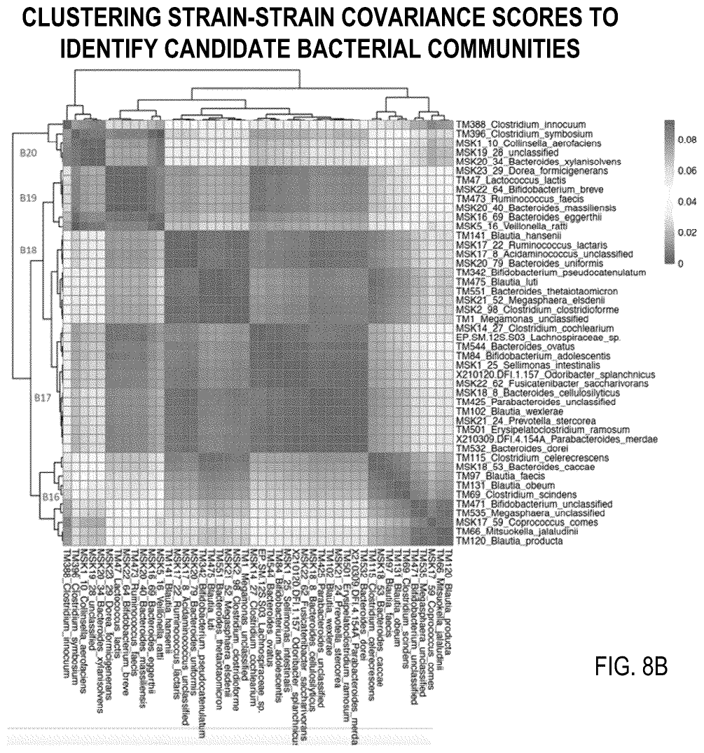

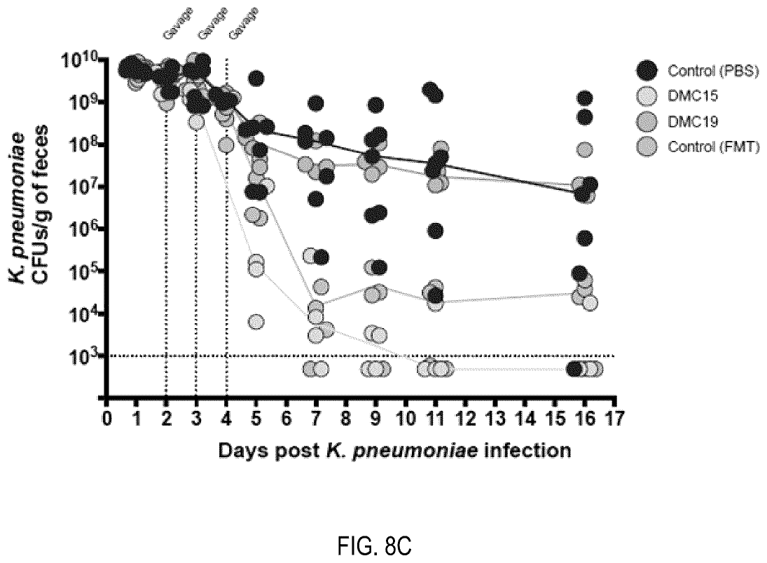

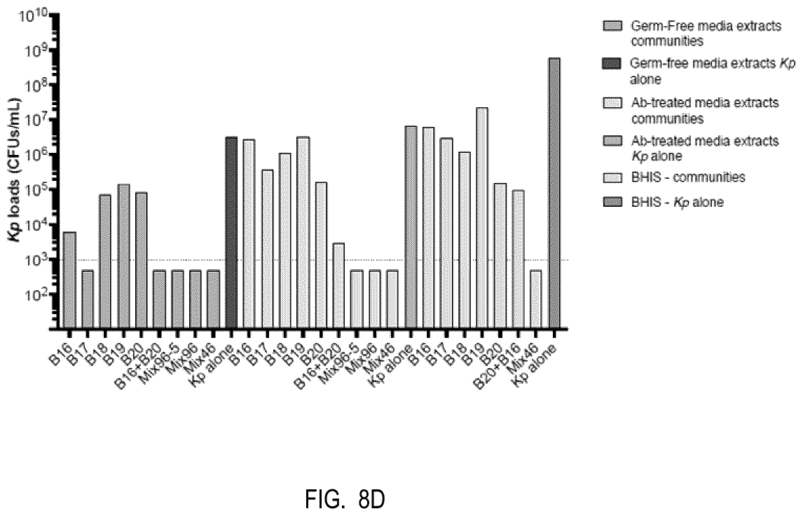

FIG. 8A-FIG. 8B show examples of experimental results on the bacterial task of suppressing drug-resistant Klebsiella pneumoniae bacteria.

In particular, FIG. 8A illustrates an example of impact scores generated by the design system for a set of bacterial strains. The bacterial strains are grouped into candidate bacterial communities, e.g., B20 (802), B16 (804), B18 (806), B17 (808), and B19 (810). The candidate bacterial communities are determined using by clustering strain-strain covariance scores for the pairs of bacterial strains.

FIG. 8B illustrates an example of clustering strain-strain covariance scores, where the strain-strain covariance scores are represented as a matrix of numerical values. It will be appreciated that, for instance, the bacterial strains in the B20 candidate bacterial community are tightly coupled, as reflected by their similar strain-strain covariance scores. B20 thus represents a sparse bacterial community with tight coupling between the bacterial strains (as reflected by the strain-strain covariance scores) and a significant correlation between the bacterial strains and performance of the bacterial task (as reflected by the impact scores).

FIG. 8C illustrates an example of experimental results for the bacterial task of suppressing drug-resistant Klebsiella pneumoniae bacteria, where specific pathogen free male mice were singly housed, fed a normal mouse chow diet, given broad spectrum antibiotics for three days, then infected with Klebsiella pneumoniae. The mice were then given three doses by both nasogastric tube orally and fecal enema of either saline (“Control (PBS)”), a fecal microbial transplant (“Control (FMT)”), or one of two bacterial communities designed using the design system: “DMC15” and “DMC19”. The DMC19 bacterial community includes the bacterial strains from the candidate bacterial communities “B20” and “B16”. The DMC19 bacterial community includes a collection of bacterial strains that share similar strain-strain covariance scores but that have relatively low impact scores. In this example, the experimental results indicate that DMC15 performs at least as well as Control (FMT), and outperforms Control (PBS) and DMC19 on the task of suppressing Klebsiella pneumoniae bacteria.

FIG. 8D illustrates an example of experimental results for the bacterial task of suppressing drug-resistant Klebsiella pneumoniae bacteria across different environments. Bacterial communities (shown on the horizontal axis) were assayed for their ability to suppress Klebsiella pneumoniae bacteria across three different environments. The “Kp” alone bars on the chart represent abundance of Klebsiella pneumoniae bacteria when bacterial communities generated by the design system are not introduced into the environment. It will be appreciated that certain bacterial communities, e.g., DMC15 (“B20+B16”) suppressed Klebsiella pneumoniae bacteria across three environments.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
training a machine learning model that is configured to process a model input that defines a bacterial community to generate a predicted task score that predicts a performance of the bacterial community in performing a bacterial task, comprising:
generating data identifying a set of bacterial communities, wherein each bacterial community comprises a plurality of bacterial strains;
obtaining, for each bacterial community, a task score for the bacterial community that represents a performance of a physically synthesized instance of the bacterial community on the bacterial task;
generating a set of training examples, wherein each training example corresponds to a respective bacterial community and comprises: (i) a training input that identifies the bacterial strains included in the bacterial community, and (ii) the task score for the bacterial community;
training the machine learning model on the set of training examples; and
identifying one or more bacterial communities for performing the bacterial task using the trained machine learning model.

2. The method of claim 1, wherein generating data identifying the set of bacterial communities comprises:
generating data identifying the set of bacterial communities using operations that encourage genetic diversity of the plurality of bacterial strains included in each bacterial community.

3. The method of claim 1, further comprising obtaining data identifying a set of bacterial strains, wherein each bacterial strain is associated with a respective feature representation;
wherein for one or more of the bacterial communities in the set of bacterial communities, generating the bacterial community comprises, at each of a plurality of iterations in a sequence of iterations:
identifying a plurality of new bacterial strains, from the set of bacterial strains, that are not currently included in the bacterial community;
determining, for each of the plurality of new bacterial strains, a distance between: (i) a feature representation of the new bacterial strain, and (ii) a respective feature representation of each of one or more bacterial strains currently included in the bacterial community; and
selecting one or more of the new bacterial strains for inclusion in the bacterial community at the iteration based on the distances.

4. The method of claim 3, wherein for each bacterial strain in the set of bacterial strains, the feature representation of the bacterial strain comprises a plurality of genetic features of the bacterial strain.

5. The method of claim 4, wherein for each bacterial strain in the set of bacterial strains, the feature representation of the bacterial strain comprises a plurality of orthologous gene group features of the bacterial strain.

6. The method of claim 1, wherein each bacterial community in the set of bacterial communities comprises bacterial strains selected from a set of bacterial strains; and
wherein the method further comprises:
obtaining a matrix representing the set of bacterial strains; and
performing dimensionality reduction on the matrix representing the set of bacterial strains.

7. The method of claim 6, wherein performing dimensionality reduction on the matrix representing the set of bacterial strains reduces a number of bacterial strains in the set of bacterial strains.

8. The method of claim 6, wherein the matrix representing the set of bacterial strains comprises a respective feature representation of each bacterial strain in the set of bacterial strains.

9. The method of claim 8, wherein for each bacterial strain in the set of bacterial strains, the feature representation of the bacterial strain comprises a plurality of genetic features of the bacterial strain.

10. The method of claim 9, wherein performing dimensionality reduction on the matrix representing the set of bacterial strains reduces a number of features in the respective feature representation of each bacterial strain.

11. The method of claim 1, wherein identifying one or more bacterial communities for performing the bacterial task using the trained machine learning model comprises:
generating a set of bacterial communities;
generating a respective predicted task score for each bacterial community of the set of bacterial communities, comprising:
processing a model input that defines the bacterial community using the machine learning model to generate the predicted task score for the bacterial community; and
identifying one or more bacterial communities for performing the bacterial task using the predicted task scores.

12. The method of claim 11, wherein identifying one or more bacterial communities for performing the bacterial task using the predicted task scores comprises:
filtering the set of bacterial communities based on the predicted task scores, comprising removing a plurality of bacterial communities having lowest predicted task scores from the set of bacterial communities;
generating a respective impact score for each bacterial strain in a set of bacterial strains using the set of bacterial communities; and
identifying one or more bacterial communities for performing the bacterial task based at least in part on the impact scores for the bacterial strains.

13. The method of claim 12, wherein generating a respective impact score for each bacterial strain in the set of bacterial strains using the set of bacterial communities comprises, for each bacterial strain:
generating a matrix representing the set of bacterial communities, wherein the matrix comprises a respective feature representation of each bacterial community in the set of bacterial communities;
processing the matrix representing the set of bacterial communities to generate a set of target vectors; and
determining the impact score for the bacterial strain based on a projection of a vector representing the bacterial strain onto the set of target vectors.

14. The method of claim 13, wherein processing the matrix representing the set of bacterial communities to generate the set of target vectors comprises:
processing the matrix representing the set of bacterial communities to generate a set of latent vectors representing axes of data variance of the matrix; and
identifying the set of target vectors as a proper subset of the set of latent vectors that have a highest statistical correlation with task scores for the bacterial task.

15. The method of claim 12, further comprising:
generating a respective strain-strain covariance score for each pair of bacterial strains in the set of bacterial strains; and
generating a set of candidate bacterial communities based on the strain-strain covariance scores;
wherein identifying one or more bacterial communities for performing the bacterial task comprises:
identifying one or more bacterial communities for performing the bacterial task based at least in part on: (i) the impact scores for the bacterial strains, and (ii) the set of candidate bacterial communities.

16. The method of claim 15, wherein generating a respective strain-strain covariance score for each pair of bacterial strains in the set of bacterial strains comprises, for each pair of bacterial strains:
determining the strain-strain covariance score for the pair of bacterial strains based on a similarity measure between: (i) a projection of a vector representing a first bacterial strain from the pair of bacterial strains onto a set of target vectors, and (ii) a projection of a vector representing a second bacterial strain from the pair of bacterial strains onto the set of target vectors.

17. The method of claim 15, wherein generating a set of candidate bacterial communities based on the strain-strain covariance scores comprises:
clustering the strain-strain covariance scores to identify a set of clusters of strain-strain covariances scores; and
generating a respective candidate bacterial community corresponding to each cluster of strain-strain covariance scores, comprising, for each cluster of strain-strain covariances scores:
generating a candidate bacterial community that includes each bacterial strain associated with a strain-strain covariance score in the cluster of strain-strain covariance scores.

18. The method of claim 15, wherein identifying one or more bacterial communities for performing the bacterial task based at least in part on: (i) the impact scores for the bacterial strains, and (ii) the set of candidate bacterial communities, comprises:
generating a respective selection score for each candidate bacterial community based at least in part on the impact scores for the bacterial strains; and
identifying one or more of the candidate bacterial communities as bacterial communities for performing the bacterial task using the selection scores for the candidate bacterial communities.

19. A system comprising:
one or more computers; and
one or more storage devices communicatively coupled to the one or more computers, wherein the one or more storage devices store instructions that, when executed by the one or more computers, cause the one or more computers to perform operations comprising:
training a machine learning model that is configured to process a model input that defines a bacterial community to generate a predicted task score that predicts a performance of the bacterial community in performing a bacterial task, comprising:
generating data identifying a set of bacterial communities, wherein each bacterial community comprises a plurality of bacterial strains;
obtaining, for each bacterial community, a task score for the bacterial community that represents a performance of a physically synthesized instance of the bacterial community on the bacterial task;
generating a set of training examples, wherein each training example corresponds to a respective bacterial community and comprises: (i) a training input that identifies the bacterial strains included in the bacterial community, and (ii) the task score for the bacterial community;
training the machine learning model on the set of training examples; and
identifying one or more bacterial communities for performing the bacterial task using the trained machine learning model.

20. One or more non-transitory computer storage media storing instructions that when executed by one or more computers cause the one or more computers to perform operations comprising:
training a machine learning model that is configured to process a model input that defines a bacterial community to generate a predicted task score that predicts a performance of the bacterial community in performing a bacterial task, comprising:
generating data identifying a set of bacterial communities, wherein each bacterial community comprises a plurality of bacterial strains;
obtaining, for each bacterial community, a task score for the bacterial community that represents a performance of a physically synthesized instance of the bacterial community on the bacterial task;
generating a set of training examples, wherein each training example corresponds to a respective bacterial community and comprises: (i) a training input that identifies the bacterial strains included in the bacterial community, and (ii) the task score for the bacterial community;
training the machine learning model on the set of training examples; and
identifying one or more bacterial communities for performing the bacterial task using the trained machine learning model.
